(19) United States
(12) Patent Application Publication
(10) Pub. No.: US 2002/0123092 A1
(43) Pub. Date: Sep. 5, 2002

(54) Regulatory elements from RP1 gene and uses thereof

(76) Inventors: Andreas Wadle, Homburg (DE); Christoph Renner, Homburg (DE); Michael Pfreundschuh, Homburg (DE)

Correspondence Address: NORMAN D. HANSON, Fulbright & Jaworski L.L.P., 666 Fifth Avenue, New York, NY, 10103, US

(21) Appl. No.: 09/790,228

(22) Filed: Feb. 21, 2001

(51) Int. Cl. C12P 21/02; C12N 5/06; C07H 21/04

(52) U.S. Cl. 435/69.1; 435/325; 435/320.1; 536/23.2

(57) ABSTRACT

The invention describes a promoter, isolated from the RP1 gene. The promoter is 10 times more active than known CMV promoters. Also described are nucleic acid molecules which are more extensive that the most active promoter described.

### FIELD OF THE INVENTION

This invention relates to nuclear regulatory elements, such as promoters and enhancers. More particularly, it relates to strong promoters and enhancers which regulate genes, such as the gene known as RP1.

### BACKGROUND AND PRIOR ART

The mechanism underlying tumor suppression are complex. One molecule involved in tumor suppression is the adenomatous polyposis coli, or “APC” tumor suppressor protein. This molecule controls the wnt signaling pathway by complexing with glycogen synthase kinase 3β (GSK-3β), axin/conductin, and β-catenin. See Polakis, Biochim. Biophys. Acta 1332: 127-147 (1997). Under normal physiological conditions, complex formation induces the rapid degradation of β-catenin in colon carcinoma cells, and defective β-catenin degradation leads to accumulation of intact β-catenin in the nucleus, where it binds to and activates Tcf-4 transcription factor.

APC protein has been characterized somewhat. For example, it is known that the region within the APC protein necessary for complex formation and initiation of β-catenin degradation is located in the N-terminal and middle portion of the protein. See Rubinfeld, et al., Science 262: 1731-1733 (1993). It is also known that APC protein has a microtubule binding domain that is clearly distinct from this first region, and is located at the C-terminal end of the molecule. Wild type, but not truncated APC protein, associates with microtubules in vivo, and promotes their assembly in vitro, as per Munemitsu, et al., Cancer Res 54: 3676-3681 (1994). This, in turn, affects important microtubule processes, such as the establishment of cell polarity (Drubin, et al., Cell 84: 335-344 (1996)), localization and organization of cell organelles, as well as intracellular vesicle trafficking. Further, APC is involved in microtubule dependent cell migration, and localization of APC is reported to be concentrated in puncta at the leading edge of prominent membrance protrusions. See Nathke, et al., J. Cell Biol 134:165-179 (1996). The mechanism by which APC protein exaggerates this function is unclear.

The highly conserved EP/RP protein family, described by Juwana, et al., Int. J. Cancer 81:275-284 (1999), incorporated by reference, is growing. Members of this family are the most likely candidates for involvement in the localization process. EB1 and RP1 protein are both characterized by ability to bind to the C terminal portion of APC and to tubulin. Both proteins localize along nuclear and cytoplasmic microtubules throughout the cell cycle. They also localize to the “plus” ends of cytoplasmic microtubules, strengthening the hypothesis that members of this family may be important for microtubule based processes.

EB/RP proteins may have a critical role in mitotic processes. This is suggested by an observed defect in yeasts, where homologues of EB1/RP1 have been characterized. See Schwartz, et al., Mol. Cell. Biol 8:2677-2691 (1997). Beinhauer et al., J. Cell Biol 139:717-728 (1997). The homologues are referred to as “BIM1p, “Ma13,” and “YEB1.” Ma13 deletion mutants show condensed chromosomes, which is a sign of mitotic delay. BIM1p deleted cells exhibit abnormally short spindles. Also, BIM1p promotes cytoplasmic microtubule dynamics specifically during the G1 phase. During this phase, microtubules in BIM1 deleted cells showed reduce dynamics, due to a slower shrinkage rate, fewer rescues and catastrophes, and more time spent in an allenuated/paused state. See Timauer, et al., J. Cell Biol. 145: 993-1007 (1999). Cells, i.e., budding yeasts, with mutated EB1/RP1 homologues also showed a delay in the cell cycle before cytokinesis. See Muhua, et al., Nature 393:487-491 (1998). These observations suggest that homologues of the EB/RP family may be a necessary component of a new, cell cycle checkpoint. Specially, the EB1/RP1 checkpoint may activate a signal to delay the cell cycle if a misoriented or defective spindle is detected.

No yeast homologue of human APC protein has been identified, rendering it difficult to study in yeast. It is postulated, however, that the homologue would play a role similar to that of the human molecule in the regulation of microtubule behavior.

The RP1 protein is described in U.S. Pat. No. 5,861,308, the disclosure of which is incorporated by reference. A 2606 base pair cDNA molecule is described, as is a 327 amino acid protein. The disclosure also refers to two additional members of the RP family, i.e., RP2 and RP3. The RP1 molecule is described as having high homology with the molecule referred to as EB2 by Su, et al., Canc. Res. 55:2972-2977 (1995), incorporated by reference. The RP proteins were discovered in activated T cells, and the pattern of RNA expression indicates that RP1 belongs to the intermediate/early gene family, as its expression is unregulated rapidly, and shut down 4-12 hours after T cell activation.

Studies on the cellular distribution pattern of EB1/RP1, characterized by specific colocalization with APC at the “plus” end of microtubules suggest that these proteins are functionally linked. What is more interesting is that the gene for APC, which is located on chromosome 5q, and the gene for RP1, which is located on chromosome 18q21, are found within chromosomal regions known to be lost or altered in a high percentage of colorectal cancers. Indeed, the localization of the RP1 gene on chromosome 18q21 (see example 1, infra), links it to a region which, according to Ogunbij, et al., J. Clin. Oncol 16: 427-433 (1988), is affected in up to 50% of all patients with colorectal cancer. Frequently, this region is altered by complete loss of the long arm of the chromosome, or deletions in the q21-23 region. Tarafa, et al., Oncogene 19:546-555 (2000), have mapped several candidate tumor suppression genes to this region, including DCC, DPC4, and MADR 2. The last two genes are members of the Smad family, which are key downstream mediators in the transforming growth factor β signalling pathway. Alterations in these genes confer resistance to TGF β, and contribute to tumorigenesis. Recent data (Papadimitrakopoulou, et al., Clin. Cancer Res 4:539-544 (1998)), suggest that there are at least two more as of yet unidentified tumor suppressor genes in this region.

Loss of heterozygosity in the long arm of chromosome 18q has been suggested as being related to poor survival, and possibly to development of metastasis. See, e.g., Jernval, et al., Br. J. Cancer 79:903-908 (1999). Increased rates of tumor recurrence is the reason for poor survival of patients who suffer loss of heterozygosity at 18q21. Clinically, loss of alleles on chromosome 18q21 serve as a marker to identify patients who are at high risk for recurrence. These factors suggest that the study of RP1 protein is of value, with respect to its role in the initiation process of colorectal cancer.

The known properties of the RP proteins have led to investigations on their trancriptional regulation. The identification and isolation of RP regulatory elements are a feature of the invention described herein, as will be seen from the disclosure which follows.

### DETAILED DESCRIPTION OF PREFERRED EMBODIMENTS

### EXAMPLE 1

These experiments describe the localization of the RP1 gene. Standard human chromosome preparations were made from phytohemagglutinin stimulated lymphocyte cultures, taken from healthy donors. A full length cDNA probe, corresponding to the coding region of the known cDNA molecule for RP1 (see emb/x94232, and U.S. Pat. No. 5,861,308, SEQ ID NO: 1, nucleotides 141-1121) (1100 base pairs) was biotinylated, using a commercially available kit, and visualized with the commercially available indicator Cy3, using standard fluorescent, in situ hybridization techniques, or “FISH.” Following the procedure, fluorescence R-banding was performed, in accordance with Zhang, et al., Hum. Genet. 103: 727-729 (1998), incorporated by reference. Docunentation of FISH and R banding results was accomplished using “ISIS” and “IKS3” software packages.

The FISH analysis indicated in more than 20 metaphase plates that the gene was found on chromosome 18. Two distinct fluorescent signals were observed. A software program, “Electronic PCR” confirmed the results.

The results are in accordance with the working draft circulated by the human genome project, which had assigned a portion of RP1's cDNA (nucleotides 2439-2591) to chromosome 18q21. See deSTS 11588-G 06091, sbSTS 55305-G 37745.

### EXAMPLE 2

These experiments describe the isolation of genomic DNA encoding RP1, and the identification of the promoter region for the RP1 gene.

A commercially available, genomic placenta λgt10 library was screened with a probe consisting of nucleotides 141-391 of RP1 cDNA (as per embs/x94232 in Genbank, see U.S. Pat. No. 5,861,308, referred to supra), and positive colonies were identified via Southern Blotting using Sambrook, et al., Molecular Cloning: A Laboratory Manual (2d. edition, 1989). A 15 kilobase pair clone was identified, which was then subdivided via restriction endonuclease Hind III, and subcloned into bacterial plasmid pBS KS⁺, which is commercially available. The resulting colonies were screened with a labelled probe consisting of the first 250 coding bases of RP1 cDNA (i.e., nucleotides ⁺141-⁺391 of SEQ ID NO: 1), via standard Southern blotting, as described supra of the positive clones identified via the screening, one 1960 base pairs long was selected for subsequent screening. The nucleotide sequence is set forth at SEQ ID NO:1. This sequence, as compared to previously disclosed materials in, e.g., U.S. Pat. No. 5,861,308, is identical over nucleotides 2163-2282 (SEQ ID NO: 1) to nucleotides 141-260 of the sequence in '308.

### EXAMPLE 3

Potential transcription start sites within this 1960 base pair fragment were identified, using a primer extension assay. In brief, total RNA was extracted from resting Jurkat cells, as well as Jurkat cells that had been stimulated with phytohemagglutinin, for anywhere from 24 to 48 hours, using standard guanidium thioisocyanate methods as exemplified by Renner, et al., J. Immunol 159:1276-1283 (1997), incorporated by reference. Then, 10 ug samples of RNA were hybridized with an end labelled primer (T4-polynucleotide kinase, α³²P-ATP), corresponding to the region that is 18 base pairs upstream of the translation start codon of the antisense strand of human RP1-cDNA, i.e.: cgcactccgg agaaggggaa c (SEQ ID NO: 2). The hybridization mixture was heated to 75° C. for 15 minutes, followed by incubation at 42° C., for forty minutes. Products were analyzed on a denatured, 6% polyacrylamide gel.

In addition, DNA sequencing reactions were carried out using the same primer, following Sambrook, et al., supra. Products were generated via cDNA extension, using M-MLV RT. These products were analyzed, as explained infra.

Three transcription start sites were identified within this fragment, at positions +1716 +1847, and +1859. The start site at position 1859 gave the strongest signal, and may represent the major transcription site.

The three DNA fragments were eluted from the polyacrylamide gel, and confirmed to be true RP1 promoter fragments via internal/nested PCR.

### EXAMPLE 4

This example sets forth a first set of experiments designed to study the activity the RP1 promoter. Two mammalian cell lines were studied, i.e., the well known, commercially available lines “293” and, “Jurkat.”

It was first necessary to assure that these lines express RP1 message endogenously, and therefore should have RP1 promoter binding elements. To do this, expression of the gene was analyzed indirectly by quantitative real time RT-PCR.

In brief, 1-5×10⁵ cells (both Jurkat and 293) were cultured, harvested, and RNA was extracted using standard methods. The RNA was eluted in a volume of 50 ul, after which an aliquot of 1 ul was reverse transcribed, using standard, commercially available products, including oligo (dT₁₅) primers, in a total volume of 20 ul. Forty cycles of amplification were then carried out, using 0.05 ul of each cDNA (20 ul reaction volume), and RP1 cDNA specific primers:

aattctatga tgctaactac gatgggaagg

(SEQ ID NO: 3)

and

gggggagttt gcatggtgag acttttttgg

(SEQ ID NO: 4)

The PCR conditions included denaturation for 10 minutes at 95° C., followed by 15 seconds at 95° C., annealing at 60° C. for 15 seconds, and amplification at 72° C. for 25 seconds, and detection using a commercially available system, i.e., the SYBR Green I detection system. Simultaneously, amplification of the β-actin gene was carried out in order to assure the quality of the isolated RNA.

Quantification of RNA was carried out via a regression linear, using a marker vector “pBI-eGFP” that had the coding region of RP1 cDNA, referred to supra, incorporated into it. For the regression, 54,321×10¹⁰, 10⁹,10⁸, 10⁷, 10⁶, and 10⁵ plasmid copies were made, via PCR. RP1 real time amplification was carried out with the two cell types, and the results were compared with the regression linear, taking the dilution rate into account. About 535 copies of mRNA were found per 293 cell (PCR cycle: 27, 46), and 945 copies per Jurkat cell (PCR cycle: 26, 79), showing that the host cells did, in fact, show endogenous RP1 expression.

### EXAMPLE 5

Different sized fragments of the RP1 gene were constructed, using standard methods. Specifically, the fragments consisted of nucleotides 751 to 1715, 751 to 2160, and 1716 to 2160 of SEQ ID NO: 1. These were then inserted into the commercially available vector pGL3. This vector lacks eukaryotic promoter and enhancer sequences. The resulting plasmids were transfected into 293 cells, and luciferase activity was determined, using standard methods.

The longest construct containing all three transcription sites (751 to 2160) was found to be 87% active, while the fragment that extended to the first site, i.e., 751 to 1715, showed no significant activity. A fragment which contained all three transcription start sites, and which extended to the coding region of the first RP1 exon (nucleotides 1716 to 2160) was the strongest fragment, and is defined as being 100% active.

Controls were also used. As a negative control, pGL3 basic vector with no insert was used, and had activity of less than 1%, while a pGL3 vector construct driven by SV40 had only 11% activity.

This experiment demonstrates that the major enchaner element of RP1 promoter is located in the fragment 1716/2160, and that the RP1 promoter has 10 times higher activity than the SV40 promoter, which is known as a strong promoter.

### EXAMPLE 6

The experiments described in this example were designed to gather more details as to the enchaner element. RP1 fragments were inserted into vector pHIVTATAluc. This is a vector in which the transcription unit synthesis of luciferase mRNA is under the control of a minimal promoter, i.e., one consisting of the HIV TATA box, and the adenovirus major late promoter element. See Thiel, et al., Gene 168:173-176 (1996), incorporated by reference. The pGL3 control vector, which contains the SV40 early promoter upstream of the luciferase gene was used as positive control. Fragments 1766/1847; 1889/2160; 1889/2061; 1889/1949; 1949/2160; 1949/2061 and 2061/2160 (relative to SEQ ID NO: 1) were inserted into pHIVTATAluc, and the recombinant vectors were transfected into 293 cells. Promoter activity was analyzed by plating 5×10⁴ cells/well in 24 well plates, and culturing these overnight, prior to transfection. In addition to the pHIVTATAluc constructs, vectors based on pGL3 and containing the constructs described in Example 5 were used. The pRL vector was used, together with the pGL3 or pHIVTATAluc vectors, either with or without inserts, in cotransfection experiments.

Cells were harvested 48 hours after transfection, and cell lysates were prepared. Light output was integrated over a 10 second period, after a 1 second read delay. Expression of firefly luciferase was normalized by the expression of the Renilla luciferase gene. All experiments were carried out in triplicate.

The results indicated that the 1889/2160 fragment was most active in the pHIVTATAluc vector system, and was defined as 100% activity. Relative to this fragment, fragments 1889/2061 and 1949/2160 showed 78% and 64% activity. The shortest fragment, i.e., 1949/2061, showed activity of 55%. All other fragments showed nearly baseline activity. The pGL3 control vector containing strong SV40 promoter was only 12% active, again indicating that RP1 is regulated by a very strong promoter. In brief, the shortest fragment, i.e., that consisting of nucleotides 1949/2061, was 10 times more active than the SV40 promoter.

### EXAMPLE 7

The preceding example suggests that the RP1 promoter is defined by nucleotides 1949-2061 of SEQ ID NO: 1. These experiments describe work designed to determine which protein or proteins interact with this DNA.

It is known that, following activation, RP1 RNA expression can be rapidly induced to high levels in Jurkat cells. See Renner, et al., J. Immunol. 159: 1276-1283 (1997).

Samples of Jurkat cells (5×10⁷), were cultured, and then stimulated with PHA for 0,4, 6,24, or 48 hours. The cells were then lysed, in 500 ul lysis buffer (50 mM KCl, 0.5% Nonidet P-40, 25 mM HEPES (N-2-hydroxyethylpiperazine-N-2-ethanesulfonic acid), pH 7.8, 1 mM phenylmethyl sulfonyl fluoride, 10 ug leupeptin/ml, 20 ug aprotonin/ml, 100 uM dithiothrectol (DTT)), on ice for 4 minutes. After 1 minute of centrifugation at 15000× g, supernatant was saved as a cytoplasmic extract. Nuclei were washed, once more, with the same volume of buffer without Nonidet-P40, and then added to 300 ul extraction buffer (500 mM KCl, 10% glycerol, 25 mM HEPES, pH 7-8, 1 mM PMSF, 10 ug leupeptin/ml, 20 ug aprotonin/ml, 100 uM DTT), and mixed several times. This was centrifuged at 14000× g for 5 minutes. Supernatant was harvested as nuclear protein extract, and was then stored, at −70° C., until used.

Double stranded DNA, consisting of nucleotides 1949-2061 of SEQ ID NO: 1, was radiolabelled, and then incubated in a 20 ul reaction mixture according to a commercially available protocol. Specifically, extracts were mixed and incubated on ice for 20 minutes in the presence of the radiolabelled probe, and then for 20 minutes at room temperature. Mixtures were analyzed on a 6% acrylamide gel that had been equilibrated with 0.5× Tris-borate EDTA buffer, at 110 volts, for 1 hour. The loaded gels were then run at 200 volts for 40 minutes, dried, and placed on film which was developed overnight at −70° C. DNA without nuclear extracts, and DNA with extracts from resting cells, were used as controls.

Maximum levels of interaction between DNA and protein were seen to have been reached after 4 hours, and then again after 24 hours. The pattern observed indicated that different protein/DNA complexes had been formed.

### EXAMPLE 8

The work set forth in example 7 was continued. Specifically, the sequence 1949/2061 was further fragmented into nucleotide sequences consisting of 1938/1964; 1972/2005, and 2006/2033, and these fragments were incubated with either nuclear Jurkat extracts, as described supra, or from commercially available HeLa nuclear extracts.

The first sequence, i.e., 1938/1964, showed no interaction with proteins from Jurkat nuclear extracts, while 1972/2005 reacted with extract from activated Jurkat cells only. The third fragment, i.e., 2006/2033, reacted with extract from both active and resting Jurkat cells. All of the fragments interacted with the HeLa extracts.

### EXAMPLE 9

Part of the sequence of fragment 1972/2005, i.e.:

tgacgtca

(SEQ ID NO: 5), is the classical cAMP responsive element, or “CRE”. As such, an assay was designed and carried out to determine if CRE binding protein, or “CREB” binds to it. To do this, fusion proteins of glutathione S-transferase and the C terminal portion of CREB, which includes its leucine dimerization domain and the region that functions as the DNA binding domain (amino acids 218-326), or a KCREB mutant (amino acids 218-326 with a mutation of R²⁸⁶→L²⁸⁶), were made by cloning the different sequences into pGEX-3X, via BgIII/EcoRI digestion. The mutated protein cannot bind to CRE (Walton, et al., Mol. Endocrinol 6(4): 647-655 (1992)). Native GST was also used as a negative control. A positive control was also used, i.e., double stranded DNA probes from the chromogranin B gene, which contains the CRE sequence referred to supra. Constructs, encoding the proteins, were introduced into protease deficient E. coli strain SG 109. The fusion proteins, and control GST, were expressed and purified in accordance with Smith, et al., Gene 67:31-40 (1988). A shift assay of the type described supra was used, where antibodies were added after 20 minutes of incubation. An assay was also carried out with CREB2 protein.

The cDNA fragment did in fact bind with both CREB proteins, but not with control proteins KCREB and GST.

The foregoing disclosure describes the features of the invention which relate, inter alia, to an isolated nucleic acid molecule which serves as a promoter for nucleic acid molecules which encode proteins, such as RP proteins, RP1 protein in particular. Such sequenced have promoter activity in the sense the term is used by those of ordinary skill in the art. See, e.g., U.S. Pat. No. 5,827,687, incorporated by reference. In brief, promoter activity means that when a sequence encoding a protein of interest is ligated downstream of the promoter in operable linkage therewith, the resulting construct, or expression vector, is expressible in, e.g., a host cell. Determination of promoter activity can be carried out by including, e.g., a gene sequence encoding a readily quantifiable protein, (i.e., a so-called “reporter gene”), such that, upon expression, the activity can be measured. More specifically, the invention relates to an isolated nucleic acid molecule comprising at least nucleotides 1949-2061 of SEQ ID NO: 1. Exemplary of such sequences are the nucleotide sequence set forth at SEQ ID NO: 1, as well as nucleic acid molecules wherein nucleotides 1949-2061 are operably linked to a nucleic acid molecule which encodes a protein, such as nucleic acid molecules that encode RP1 protein. Exemplary of such sequences is, e.g., SEQ ID NO: 1 of U.S. Pat. No. 5,861,308. Additional examples of such encoding nucleic acid molecules include, e.g., tumor rejection antigen precursors, such as members of the MAGE family (see, e.g., U.S. Pat. No. 5,342,774), the GAGE, BAGE, TAGE, and RAGE molecules, other full length molecules such as NY-ESO-1, Melan-A, MART-1, and so forth. The promoters of the invention can also be placed in operable linkage with molecules that encode portion of full length molecules, such as tumor rejection antigens of the type described in, e.g., U.S. Pat. Nos. 5,405,940; 6,025,470 etc. Other molecule portions, such as immunogenic domains, enzymatically active portions of molecules, MHC-Class II binding peptides, antisense DNA antisense RNA, sequences encoding decoys, ribosomes etc., can also be encoded by the nucleic acid molecule placed in operable linkage with the promoter. More than one peptide can be encoded by the linked nucleic acid molecule as per, e.g., polytope constructions. Proteins and peptides of interest in other areas, such as therapeutically useful proteins (e.g., erythropoietin, GM-CSF, interleukins, cytokines, growth factors, etc), can be manufactured by linking nucleic acid molecules which encode the proteins to the promoter of the invention. Further, the promoters of the invention can be incorporated into, e.g., reagent kits where the promoter constitutes one separate portion of the reagent kit, together with additional portions of further reagents, such as a coding sequence, etc. Diagnostically useful proteins, such as recombinant antibodies, industrially useful proteins such as enzymes, may all be made in the same way, using the expression vector constructs of the invention. The isolated recombinant cells of the invention, which have been transformed or transfected with the expression vector of the invention, are also a part of the invention. As will be seen, supra, the RP1 promoter described herein functions in the same way the CMV promoter functions, but is an order of magnitude stronger. The CMV promoter is well known as a standard promoter for various constructs which encode proteins and is considered by some to be the “gold standard” by which other promoters are judged. See, e.g., Foecking, et al., Gene 45(1): 101-105 (1986), Boshart, et al., Cell 41(2): 521-30 (1985), both of which are incorporated by reference. Any such construct can be modified by replacing the CMV promoter with the RP1 promoter described herein.

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

We claim:

1. An isolated nucleic acid molecule which has promoter activity, comprising at least nucleotides 1949 to 2061 of SEQ ID NO:1, and no more than the nucleotide sequence set forth at SEQ ID NO:1.

2. The isolated nucleic acid molecule of claim 1, comprising at least nucleotides 1949 to 2061 of SEQ ID NO:1, and no more than nucleotides 1889 to 2160 of SEQ ID NO:1.

3. The isolated nucleic acid molecule of claim 1, comprising nucleotides 1889 to 2061 of SEQ ID NO:1.

4. The isolated nucleic acid molecule of claim 1, comprising nucleotides 1949 to 2160 of SEQ ID NO:1.

5. The isolated nucleic acid molecule of claim 1, comprising nucleotides 1949 to 2061 of SEQ ID NO: 1.

6. The isolated nucleic acid molecule of claim 1, comprising nucleotides 751 to 2160 of SEQ ID NO: 1.

7. The isolated nucleic acid molecule of claim 1, comprising nucleotides 751 to 1715 of SEQ ID NO:1.

8. The isolated nucleic acid molecule of claim 1, comprising nucleotides 1716 to 2160 of SEQ ID NO:1.

9. The isolated nucleic acid molecule of claim 1, comprising the nucleotide sequence of SEQ ID NO:1.

10. Expression vector comprising the isolated nucleic acid molecule of claim 1, in operable linkage with a nucleic acid molecule that encodes a protein.

11. The expression vector of clam 10, wherein said protein is an RP1 protein.

12. Isolated, recombinant cell comprising the isolated nucleic acid molecule of claim 1.

13. Isolated recombinant cell comprising the expression vector of claim 10.

14. A method for expressing a nucleotide sequence of interest, comprising inserting the expression vector of claim 10 into a host cell, and culturing said cell to expires said protein.

15. A method for expression a nucleotide sequence of interest, comprising inserting the isolated nucleic acid cell containing said nucleotide sequence of interest to express it. Molecule of claim 1 upstream of said nucleotide sequence of interest and in operable linkage therewith, and culturing a cell containing said nucleotide sequence of interest to express it.


SEQUENCE LISTING (5 sequences)

SEQ ID NO: 1 - DNA, 3583 bp, Homo sapiens
  unsure 964, 1025, 3242, 3274, 3329, 3425, 3431, 3432, 3433,3434, 3435, 3436, 3437, 3438, 3442, 3445, 3457, 3463, 3465, 3514,3520, 3551, 3554, 3561, 3565, 3567, 3570, 3571, 3572: unknown
aagcttggtgtgtatctttgatacttagatattcatctgtgtaccttgtaagtcttaggtcagaattggatctgattgtataagaaaaaaaatccttatgtaaagtgagatgtggacgaaggaatcaacttgtgcactgtggactaactggagaagcaaaagataaaattattaaaataatagcccaagaggaaagtataaaataggaaggaaaataagtagggccccttgtttgagattatccttaccatctgggttgtaaaacatttgattctaattccagaaacttgataccagaaataacttattaccagggacatatgctgaagaattgtcaggaatgacaaagtgtttacttgtttttaatatctgtatcacaaaatgataaagctgataaaccatacagtgatgtgaaggagtgagtcatagcttaatactttatgacacatatatttatgctgtatcttttacaccccaattcttcccttttcaccctataaaaggtcagagaccactgctgataagcaaagaatccttcaaaatctctcctggctgggcgtggtggctcatgcctgtaatcctagcactttggggcgggtggactgcctgagctcaggagttcaagaccagcctgagcaactcggtgaaaccctgtctctactaaaatacaaaaattagctgggtgtggcggcgtgcgcctgttatcccacctactcaggaggctgaggcaggagaattactagaacccaggaggcggaggttgcagtgagctgagattgcgccactgcactccagcctgggtgacagagcgagactctgcctctaaaaaataataataaaaaaaaaatctttcctctctggaggacagtactggaacatatgcagggtctttcagtttataatcctaatcccgtagactgcacagtgctagcaattctgttcacagttatcttttcacaacagncatcttggaagtacttgattcttaccttgatttataatatttattgagctcacattacagntcattactgtccagtcacattgataaaaccatgtcacttggaagctccagttcaccactcctagctgtgtgaccttaagcagtttacttaatcttactagtttactcatctttaaaatgggaataataatagtatctgtcacatagccaatatatttgaaagcggtattgcaaggatatagaacagcttgaatgttaagcactttcaaatactaagtgaggtgatcacttttgtaattcaaatggaaatagaataaatgtcaatgaattaatgatctgtatcaggaggcagcataaagaatttgaattggagggttttgggtaactggtaaaatggtggttatcttttttttaaaagaaaaaccctctttggggattcctttgacagatgatctcttaatttgccatctttaatcctcacattattaaaggctacattgattagaaggaaatacagcttccttgtaggtttttagctttttggcttgtttttggggagatggttgctgctatttggagaaagggagaaaggcagaaattttaaccttagtctgataccttttctacaagctggccaccaggtcctttttaggtttttctgaaacctgaccaagtttaattgtcatggcataaaaacttggtattgaaggagttaaagggtctcccacacccctttgcatgaatcgttcttttgtgtacacagctgcagtggcagcacgggtcggaaaggaaatggttagaggaagagctggttttcagacaaggcgagtctgcgaggggggctgcagggtggcctggggttttacatgccagatgtaggcctgccccggtgcctgcctggagctgctggcgtggtcacgccgcgaccctgtgcgaattgaggcgaggtgatgagttagcgggttgccatggcaacaggctggccacgtgaccagggtgctgctgccggcgctctgacgtcagctgccagagggcggggccgcaggagggcggggcgcgagcgagagctgggagaaggcagtgagcgagcaggcggcaggcacggtccgtgcggagcaggcgagcgagcgggaagacgcagccaccttcctcaccagccagcccacagcggtttgttccccttctcgggagtgcgccaatgcctgggcgacccaaaccctgtccccaaatggcgagaacaacaacgacatcatccaggataataacgggaccatcattcctttccggaagcacacagtgcgcggggagcgttcctacaggtaatggggctggcacgagagcagcgccggggaccgccgtgagggcgcgcggaaatccagcccgttatataatggagcagacactgcctgcgaacccctgctgcaggcatgcatttgtgaaggcggttgtgattatttggtatctgtttccatgtgtcatgttgggtttgcattgaggctccgcgactgcagatggaaagcattttgtgaataggtttgctgccttcgaggggtctccctccatctctttccttccacactgataaaaatgcatgctgttttcccccgctgtccaaagggagacaaggcagtagcacttcctttcctccttcctttagattttaaggagccattaggtagggtggcagtgccaaaaggcrgagaacggggggggggggggagaaacgtktgcgcttaacccggagagtgtgcagggaagatggaatcctctagattaagctgcagtctgtgatgttaaaccgccacgattgatggggctttgtaagagmttctgctcgctgattggtgcagggattgacagcagaggtgatagtgtcagacattcaccaaaattttctccatagaaccctaggtttattagaaagagactggacttcaagagacttgaaaaaaactgggcgcgtgttatgcgacttttctaaggagttatggccaccscttgggtgggggagcctttccttcttatttacartaggaaggtgattccyggtcttttkctgaggtcttttttgaggatgggacttatcytggtcattgtattgtgagccttttgattaatgagttggccygtacggttggcatcatttttacattaacatatctkgcaatatgttccmgtagataagctctattaaaattgtttgcattgtttgataaggataacaatgtaatggagaatgcagctagagtcttatgcagctcaaggccmgacttgaagngagactgatagcttcggtcatatctccyctgnagttctcttgtactggggtagttatttctaatgttaccctgataaacgctaacantgctttttgctttgaggcagagcacacatacccctaaagcgcatcccctcagctgatgagcacacgaatactctttcttccttttagaccgcactnacatcnnnnnnnntttnggnagatattttttntaaatntncaacccaatagcccgaagccctcttcctcccttgccaagaatcattttnctgccnaaaaaaaatattactacttcccctcatcttncantcataancttngnttnnntgctcygccac

SEQ ID NO: 2 - DNA, 21 bp, Homo sapiens
cgcactccggagaaggggaac

SEQ ID NO: 3 - DNA, 30 bp, Homo sapiens
aattctatgatgctaactacgatgggaagg

SEQ ID NO: 4 - DNA, 30 bp, Homo sapiens
gggggagtttgcatggtgagacttttttgg

SEQ ID NO: 5 - DNA, 8 bp, Homo sapiens
tgacgtca